(19) United States
(12) Patent Application Publication
(10) Pub. No.: US 2006/0247427 A1
(43) Pub. Date: Nov. 2, 2006

(54) Process to obtain 6-O-methylerythromycin a (clarithromycin)_form II

(75) Inventors: Pandurang Balwant Deshpande, Vadodara (IN); Rohit Ravikant Soni, Vadodara (IN); Mahadeo Maruti Thorat, Vadodara (IN); Bhavisha Nimesh Dave, Vadodara (IN); Anand Kumar Pandey, Vadodara (IN)

Correspondence Address: HAMRE, SCHUMANN, MUELLER & LARSON, P.C., P.O. BOX 2902, MINNEAPOLIS, MN, 55402-0902, US

(73) Assignee: ALEMBIC LIMITED, Vadodara (IN)

(21) Appl. No.: 11/491,735

(22) Filed: Jul. 24, 2006

(30) Foreign Application Priority Data
Aug. 31, 2005 (IN) 1050/MUM/2005

(51) Int. Cl. C07H 17/08

(52) U.S. Cl. 536/7.2

(57) ABSTRACT

The present invention provides a process for the preparation of 6-O-methylerythromycin A Form II comprising treating 6-O-methylerythromycin A with organic acid selected form trifluoroacetic acid, para-toluene sulphonic acid, oxalic acid or acetic acid and converting it into an organic salt of 6-O-methylerythromycin A, which can be neutralized by base to give 6-O-methylerythromycin A Form II.

### FIELD OF INVENTION

The present invention relates to organic salt of 6-O-methylerythromycin A of formula (II), and its use in the process for preparing 6-O-methylerythromycin A Form II with high purity and yield. 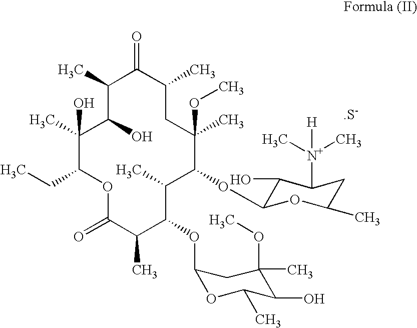
wherein S is organic acid selected from group of trifluoroacetic acid, para-toluene sulphonic acid, oxalic acid or acetic acid.

### BACKGROUND OF THE INVENTION AND PRIOR ART

Clarithromycin is a semi-synthetic macrolide antibiotic of formula (I), chemically know as 6-O-methylerythromycin A. It is a semi synthetic macrolide antibiotic which exhibits strong antibacterial activity towards a wide range of bacteria inclusive of gram positive bacteria, some gram negative bacteria, anaerobic bacteria, mycoplasma, chlamydia and helicobacter pylori, and because of its high stability in acidic environment of the stomach, it can be orally administered to treat respiratory organ diseases, and also to prevent recurrence of ulcer when used in combination with other medicine.

Various forms of Clarithromycin are reported. These include Clarithromycin Form I, II, 0, III and IV. These crystal forms and their process for preparation are described in various patents viz. U.S. Pat. No. 5,858,986, U.S. Pat. No. 5,844,105, U.S. Pat. No. 5,945,405, U.S. Pat. No. 6,627,743, U.S. Pat. No. 6,599,884, U.S. Pat. No. 6,515,116, U.S. Pat. No. 6,444,796, etc. Form II is thermodynamically more stable than Form I and is used in the drug formulations currently available in market. Form 0 is solvated form of Clarithromycin having an incorporated crystallizing solvent molecules of solvent selected from ethanol, isopropanol, isopropyl acetate and tetrahydrofuran. Form III is acetonitrile solvate of Clarithromycin.

The process of preparing Form II as disclosed in U.S. Pat. No. 5,844,105 involves crystallization or recrystallization of Clarithromycin from solvent selected from the group of: (i) an alkanol of from 1 to 5 carbon atoms, provided said alkanol is not ethanol or isopropanol, (ii) a hydrocarbon of from 5 to 12 carbon atoms, (iii) a ketone of from 3 to 12 carbon atoms, (iv) a carboxylic ester of from 3 to 12 carbon atoms, provided said carboxylic ester is not isopropyl acetate, (v) an ether of from 4 to 10 carbon atoms, (vi) benzene, (vii) benzene substituted with one or more substituents selected from the group consisting of alkyl of from one to four carbon atoms, alkoxy of from one to four carbon atoms, nitro, and halogen, (viii) a polar aprotic solvent, (ix) a compound having the formula HNR¹ R² wherein R¹ and R² are independently selected from hydrogen and alkyl of one to four carbon atoms, provided that R¹ and R² are not both hydrogen, (x) water and a water miscible solvent selected from the group consisting of a water miscible organic solvent and a water miscible alkanol, (xi) methanol and a second solvent selected from the group consisting of a hydrocarbon of from 5 to 12 carbon atoms, an alkanol of from 2 to 5 carbon atoms, a ketone of from 3 to 12 carbon atoms, a carboxylic ester of from 3 to 12 carbon atoms, an ether of from 4 to 10 carbon atoms, benzene, and benzene substituted with one or more substituents selected from the group consisting of alkyl of from one to four carbon atoms, alkoxy of from one to four carbon atoms, nitro, and halogen, and (xii) a hydrocarbon of from 5 to 12 carbon atoms and a second solvent selected from the group consisting of a ketone of from 3 to 12 carbon atoms, a carboxylic ester of from 3 to 12 carbon atoms, an ether of from 4 to 10 carbon atoms, benzene, benzene substituted with one or more substituents selected from the group consisting of alkyl of from one to four carbon atoms, alkoxy of from one to four carbon atoms, nitro, and halogen, and a polar aprotic. However this method of crystallization or recrystallization does not enhance the purity of crude Clarithromycin.

U.S. Pat. No. 5,858,986 and U.S. Pat. No. 5,945,405 discloses process for preparation of Clarithromycin Form II crystals which involves heating Form 0 or Form I crystals under vacuum at a temperature ranging from 70 to 110° C. for a prolonged period of time to prepare Form II crystals, but this method has the problem of low productivity and high cost and also does not enhance the purity of Clarithromycin.

Therefore there is a need to develop a high yield process for preparing 6-O-methylerythromycin A Form II having high purity, which is simple, easy to handle and cost effective on commercial scale.

### OBJECT OF THE INVENTION

Accordingly, it is the primary object of the present invention to provide a process which results in increased yield and purity of 6-O-methylerythromycin A Form II.

According to the primary object of the invention there is provided a novel intermediate which is organic acid salt of 6-O-methylerythromycin A of formula (II).

Accordingly yet another object of the invention is to provide process for preparation of novel intermediate which is organic acid salt of 6-O-methylerythromycin A of formula (II).

Yet another object of invention is to provide process for preparing 6-O-methylerythromycin A Form II by using novel intermediate which is organic acid salt of 6-O-methylerythromycin A of formula (II), which results in high yield and purity of the product.

### SUMMARY OF THE INVENTION

Accordingly to the object of the invention, one of the embodiments of the present invention provides method of preparing 6-O-methylerythromycin A Form II comprising:

(a) treating 6-O-methylerythromycin A of formula (I) with organic acid selected from group of trifluoroacetic acid, para-toluene sulphonic acid, oxalic acid or acetic acid, in a solvent to give organic acid salt of 6-O-methyl Erythromycin A of formula (II) 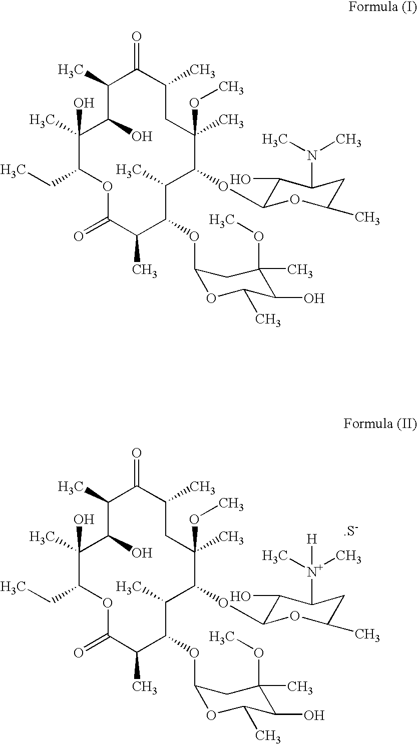
wherein S is organic acid selected from group of trifluoroacetic acid, para-toluene sulphonic acid, oxalic acid or acetic acid.

(b) neutralizing the organic acid salt of 6-O-methyl Erythromycin A of formula (II) with base in solvent to give 6-O-methylerythromycin A Form II crystals.

Another embodiment of the present invention provides process for preparation of the organic acid salt of 6-O-methyl Erythromycin A of formula (II) comprising of treating 6-O-methyl Erythromycin A of formula (I) with organic acid selected from group of trifluoroacetic acid, para-toluene sulphonic acid, oxalic acid or acetic acid, in a solvent.

Yet another embodiment of the present invention provides novel intermediate which is organic acid of 6-O-methyl Erythromycin A of formula (II): 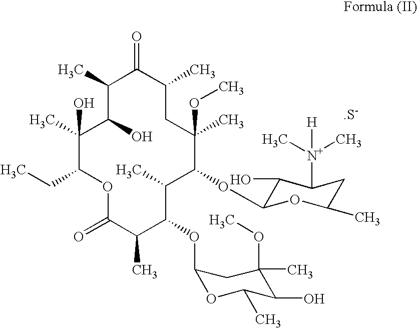
wherein S is organic acid selected from group of trifluoroacetic acid, para-toluene sulphonic acid, oxalic acid or acetic acid.

Still another embodiment of the present invention provides process of preparing 6-O-methylerythromycin A Form II, which is high yielding, results in improved purity of the product, is easy to handle and cost effective, by using novel intermediate which is organic acid salt of 6-O-methylerythromycin A of formula (II).

### DETAILED DESCRIPTION OF THE INVENTION

The term “Clarithromycin” or “6-O-methylerythromycin A” as used herein refers to Clarithromycin of any purity and may be solid, semisolid, or in form of syrup or it may exists in any crystalline forms in pure state of mixtures of Form I, II and 0.

Clarithromycin used in the process of present invention can be prepared by any of methods disclosed in prior art.

6-O-methylerythromycin A Form II as referred herein is similar to 6-O-methylerythromycin A Form II as referred in U.S. Pat. No. 5,945,405 and U.S. Pat. No. 5,844,105, in terms of crystalline nature specifically powder X-ray diffraction pattern and peaks.

It has been surprisingly found by the inventors of the present invention that 6-O-methylerythromycin A Form II can be prepared in high yield and purity by converting crude 6-O-methylerythromycin obtained by any of the process of preparation well known in art, to an organic acid salt and subsequently converting the organic acid salt to 6-O-methylerythromycin A Form II.

The process of preparing 6-O-methylerythromycin A Form II comprises of following two steps:

(a) treating 6-O-methylerythromycin A of formula (I) with organic acid selected from group of trifluoroacetic acid, para-toluene sulphonic acid, oxalic acid or acetic acid, in a solvent to give organic acid salt of 6-O-methyl Erythromycin A of formula (II) 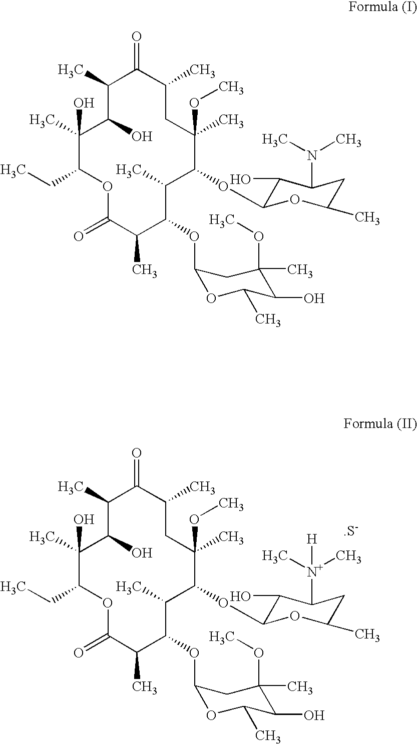
wherein S is organic acid selected from group of trifluoroacetic acid, para-toluene sulphonic acid, oxalic acid or acetic acid.

(b) neutralizing the organic acid salt of 6-O-methyl Erythromycin A of formula (II) with base in solvent to give 6-O-methylerythromycin A Form II crystals.

In accordance with step (a) of the present invention, the organic acid salt of 6-O-methyl Erythromycin A of formula (II) is prepared by treating 6-O-methylerythromycin A with organic acid selected from group of trifluoroacetic acid, para-toluene sulphonic acid, oxalic acid or acetic acid in a solvent and then isolating the product by conventional methods.

Crude Clarithromycin is suspended in organic solvent at temperature ranging from room temperature to the boiling point of the solvent. Then organic acid is added to the slurry and stirred for about 10 minutes to about 5 hours, preferably for about 1 hour to about 4 hours and most preferably for about 3 hours. The organic acid salt thus formed is isolated by conventional methods such as filtration or centrifugation.

The solvents used can be selected from the group comprising of (i) C₁₋₆ alkanol, (ii) C₃₋₆ ketone, (iii) C₃₋₈ carboxylic ester, (iv) C₁₋₆ nitrile, (v) C₄₋₁₀ ether, (vi) benzene, (vii) benzene substituted with at least one of the substituents selected from C₁₋₃ alkyl, C₁₋₃ alkoxy, nitro and halogen, (viii) C₅₋₁₂ hydrocarbon, (ix) C₁₋₄ nitroalkane, (x) aprotic polar solvent, (xi) chlorinated hydrocarbons, (xii) water, and (xiii) a mixture thereof. The preferred examples of solvents include methanol, ethanol, propanol, isopropanol, butanol, isobutanol, tert-butanol, pentanol, hexanol, ethylene glycol, 1,2- or 1,3-propylene glycol, acetone, methyl ethyl ketone, 2-pentanone, 3-pentanone, methyl isobutyl ketone, methyl acetate, ethyl acetate, propyl acetate, isobutyl acetate, methyl propionate, acetonitrile, propionitrile, ethyl ether, isopropyl ether, methyl tert-butyl ether, tetrahydrofuran, dioxane, ethylene glycol dimethyl ether, ethylene glycol diethyl ether, diethylene glycol dimethyl ether, benzene, toluene, xylene, chlorobenzene, nitrobenzene, anisole, pentane, hexane, heptane, cyclohexane, nitromethane, nitroethane, nitropropane, N,N-dimethyl formamide, N,N-dimethyl acetamide, dimethyl sulfoxide, sulfolane, dichloromethane, dichloroethane, water and a mixture thereof, wherein preferred are acetone, methyl isobutyl ketone, dichloromethane or mixtures thereof.

After isolating the organic acid salt of 6-O-methyl Erythromycin A of formula (II), it may be further purified by recrystallization from solvent as specified above. If required carry out repeated crystallization from solvent to get the desired purity of the compound.

According to one of the preferred embodiments, crude Clarithromycin is suspended in acetone. Trifluoroacetic acid is added to the slurry and stirred for about three hours at about 25-30° C. After the reaction is complete the trifluoroacetate salt of 6-O-methylerythromycin of formula (III), is filtered and washed. The organic salt thus obtained is crystallized from ethyl acetate and used for step (b). 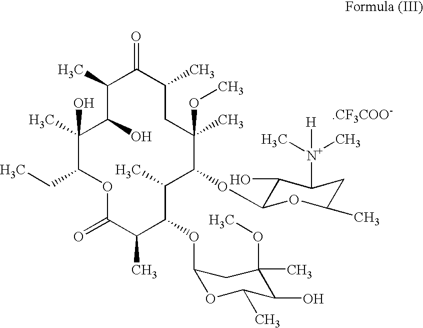
In accordance with step (b) of the present invention 6-O-methylerythromycin A Form II is prepared by neutralizing the organic acid salt of 6-O-methyl Erythromycin A of formula (II), with base in a solvent.

The organic acid salt of 6-O-methyl Erythromycin A of formula (II) is dissolved in a solvent in temperature ranging from room temperature to the boiling point of the solvent. The reaction mixture is then basified with base till pH in the range of 7 to 12 is obtained, more preferably in the range of 9 to 11. Then the reaction mixture can be stirred for a period of about 10 minutes to 5 hours, more preferably at for about an hour at a temperature range from about room temperature to about boiling point of the solvent. The product thus obtained is isolated by conventional methods such as filtration or centrifugation and dried to give pure 6-O-methylerythromycin A Form II.

The solvent used in step (b) of the invention is selected from group comprising of (i) C₁₋₆ alkanol, (ii) C₃₋₆ ketone, (iii) C₁₋₆ nitrile, (iv) diether and cyclic ether, (v) aprotic polar solvent, (vi) water, and (vii) a mixture thereof. The preferred examples of solvents include methanol, ethanol, propanol, isopropanol, butanol, isobutanol, tert-butanol, pentanol, hexanol, ethylene glycol, 1,2- or 1,3-propylene glycol, acetone, methyl ethyl ketone, 2-pentanone, 3-pentanone, methyl isobutyl ketone, acetonitrile, tetrahydrofuran, dioxane, ethylene glycol dimethyl ether, ethylene glycol diethyl ether, diethylene glycol dimethyl ether, N,N-dimethyl formamide, N,N-dimethyl acetamide, dimethyl sulfoxide, sulfolane, water and a mixture thereof, wherein preferred are ethanol, water and mixtures thereof.

The base used in step (b) is selected from the group comprising of alkali and alkaline metal hydroxide, alkali and alkaline metal carbonate, alkali and alkaline metal bicarbonate, NR¹R²R³ (wherein, R¹, R² and R³ are each independently hydrogen or C₁₋₄ alkyl), and a mixture thereof. The preferred examples of base include sodium hydroxide, potassium hydroxide, calcium hydroxide, magnesium hydroxide, sodium bicarbonate, potassium bicarbonate, sodium carbonate, potassium carbonate, cesium carbonate, ammonia, triethyl amine, and the like and the most preferred is sodium hydroxide.

According to one of the preferred embodiments the trifluoroacetate salt of 6-O-methylerythromycin A of formula (III) is dissolved in mixture of 1:1 ethanol-water and heated to about 40° C. The solution is basified with 10% aqueous sodium hydroxide solution, stirred at about 40° C. for about an hour, filtered and dried in conventional manner.

The 6-O-methylerythromycin A Form II shows an increase in purity compared to crude Clarithromycin. The process of the present invention is simple, easy to handle, cost effective and shows increase in yield.

The examples given below illustrate the process of the present invention but do not intend to limit the scope of the present invention.

### EXAMPLE 1

### Preparation of trifluoroacetate salt of Clarithromycin

10 g crude Clarithromycin was suspended in 30 ml acetone. 1 ml trifluoroacetic acid was added to it and the slurry was stirred for about 3 hours at about 25-35° C. The solid was filtered and washed with acetone to get trifluoroacetate salt of Clarithromycin. This salt was recrystallized twice from ethyl acetate to obtain trifluoroacetate salt of Clarithromycin having purity more than 98% (determined by HPLC).

### EXAMPLE 2

### Preparation of para-toluene sulphonate salt of Clarithromycin

10 g crude Clarithromycin was suspended in 30 ml dichloromethane. 2.4 g para-toluene sulphonic acid was added to it and the slurry was stirred for about 3 hours at about 25-35° C. The solid was filtered and washed with dichloromethane to get para-toluene sulphonate salt of Clarithromycin. This salt was recrystallized twice from acetone to obtain para-toluene sulphonate salt of Clarithromycin having purity more than 98% (determined by HPLC).

### EXAMPLE 3

### Preparation of oxalate salt of Clarithromycin

10 g crude Clarithromycin was suspended in 30 ml methyl isobutyl ketone. 1.6 g oxalic acid was added to it and the slurry was stirred for about 3 hours at about 25-35° C. The solid was filtered and washed with methyl isobutyl ketone to get oxalate salt of Clarithromycin. This salt was recrystallized from ethyl acetate followed by second recrystallization from acetone to obtain oxalate salt of Clarithromycin having purity more than 98% (determined by HPLC).

### EXAMPLE 4

### Preparation of 6-O-methylerythromycin A Form II

5 g of organic acid salt (from Example 1, 2 or 3) was dissolved in 50 ml mixture of ethanol-water (1:1) and heated to about 40° C. Additional 25 ml of water is added to the solution and it is basified with 10% aqueous sodium hydroxide solution till pH 9 to 11. The slurry is then stirred at about 40° C. for about an hour and filtered. The wet cake is washed with hot water and dried at 50° C. in vacuum to get pure 6-O-methylerythromycin A Form II.

1. A process of preparing 6-O-methylerythromycin A Form II comprising:
(a) treating 6-O-methylerythromycin A of formula (I) with organic acid selected from group of trifluoroacetic acid, para-toluene sulphonic acid, oxalic acid or acetic acid, in a solvent to give organic acid salt of 6-O-methyl Erythromycin A of formula (II) 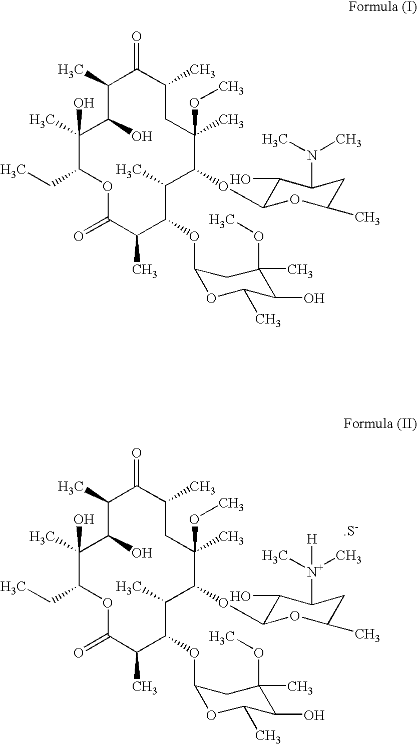
wherein S is organic acid selected from group of trifluoroacetic acid, para-toluene sulphonic acid, oxalic acid or acetic acid.
(b) neutralizing the organic acid salt of 6-O-methyl Erythromycin A of formula (II) with base in solvent to give 6-O-methylerythromycin A Form II crystals.

2. A process as claimed in claim 1, wherein said organic acid used in step (a) is trifluoroacetic acid.

3. A process as claimed in claim 1, wherein solvent used in step (a) is selected from the group comprising of (i) C₁₋₆ alkanol, (ii) C₃₋₆ ketone, (iii) C₃₋₈ carboxylic ester, (iv) C₁₋₆ nitrile, (v) C₄₋₁₀ ether, (vi) benzene, (vii) benzene substituted with at least one of the substituents selected from C₁₋₃ alkyl, C₁₋₃ alkoxy, nitro and halogen, (viii) C₅₋₁₂ hydrocarbon, (ix) C₁₋₄ nitroalkane, (x) aprotic polar solvent, (xi) chlorinated hydrocarbons, (xii) water, and (xiii) a mixture thereof.

4. A process as claimed in claim 3, wherein solvent is selected from acetone, methyl isobutyl ketone, dichloromethane or mixtures thereof.

5. A process as claimed in claim 1 wherein, the solvent used in step (b) is selected from the group comprising of (i) C₁₋₆ alkanol, (ii) C₃₋₆ ketone, (iii) C₁₋₆ nitrile, (iv) diether and cyclic ether, (v) aprotic polar solvent, (vi) water, and (vii) a mixture thereof.

6. A process as claimed in claim 5 wherein, said solvent is ethanol, water or mixtures thereof.

7. A process claimed in claim 1 wherein, the base use in step (b) is selected from the group comprising of alkali and alkaline metal hydroxide, alkali and alkaline metal carbonate, alkali and alkaline metal bicarbonate, NR¹R²R³ (wherein, R¹, R² and R³ are each independently hydrogen or C₁₋₄ alkyl), and a mixture thereof.

8. A process as claimed in claim 7, wherein the base is selected from group consisting of sodium hydroxide, potassium hydroxide, calcium hydroxide, magnesium hydroxide, sodium bicarbonate, potassium bicarbonate, sodium carbonate, potassium carbonate, cesium carbonate, ammonia, triethyl amine, and the like.

9. A process as claimed in claim 8, wherein the base is sodium hydroxide.

10. A novel organic acid salt of 6-O-methyl Erythromycin A of formula (II): 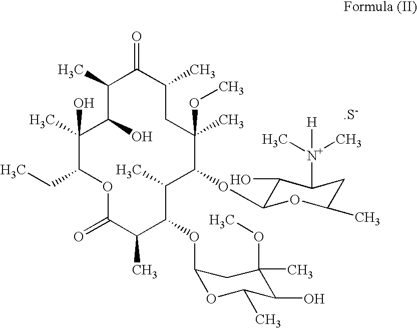
wherein S is organic acid selected from group of trifluoroacetic acid, para-toluene sulphonic acid, oxalic acid or acetic acid.

11. A compound which is trifluoroacetate salt of 6-O-methyl Erythromycin A of formula (III):

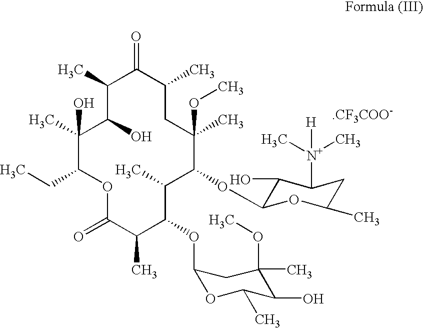

12. A process for preparation of the organic acid salt of 6-O-methyl Erythromycin A of formula (II) comprising of treating 6-O-methyl Erythromycin A of formula (I) with organic acid selected from group of trifluoroacetic acid, para-toluene sulphonic acid, oxalic acid or acetic acid, in a solvent. 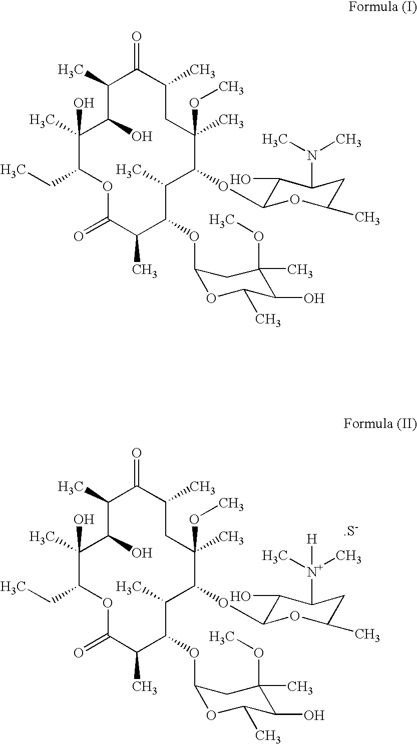
wherein S is organic acid selected from group of trifluoroacetic acid, para-toluene sulphonic acid, oxalic acid or acetic acid.

13. A process as claimed in claim 12, wherein said organic acid is trifluoroacetic acid.

14. A process as claimed in claim 12, wherein solvent used is selected from the group comprising of (i) C₁₋₆ alkanol, (ii) C₃₋₆ ketone, (iii) C₃₋₈ carboxylic ester, (iv) C₁₋₆ nitrile, (v) C₄₋₁₀ ether, (vi) benzene, (vii) benzene substituted with at least one of the substituents selected from C₁₋₃ alkyl, C₁₋₃ alkoxy, nitro and halogen, (viii) C₅₋₁₂ hydrocarbon, (ix) C₁₋₄ nitroalkane, (x) aprotic polar solvent, (xi) chlorinated hydrocarbons, (xii) water, and (xiii) a mixture thereof.

15. A process as claimed in claim 14, wherein solvent is selected from acetone, methyl isobutyl ketone, dichloromethane or mixtures thereof.

16. Use of novel organic acid salt of 6-O-methyl Erythromycin A of formula (II): 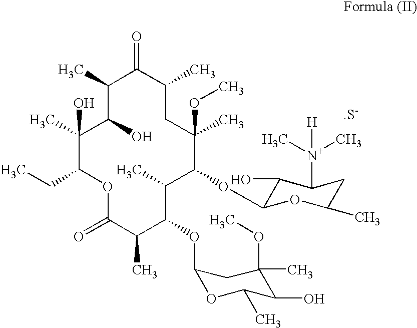
wherein S is organic acid selected from group of trifluoroacetic acid, para-toluene sulphonic acid, oxalic acid or acetic acid, in synthesis of 6-O-methylerythromycin A Form II.

17. A process for the preparation of 6-O-methylerythromycin A Form II such as herein described in accompanying text, description and examples.
